(19) United States
(12) Patent Application Publication
(10) Pub. No.: US 2006/0121491 A1
(43) Pub. Date: Jun. 8, 2006

(54) Partially degenerate oligonucleotide standards and methods for generating the same

(76) Inventors: Paul K. Wolber, Los Altos Hills, CA (US); Anthea Dokidis, Palo Alto, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 11/001,867

(22) Filed: Dec. 2, 2004

(51) Int. Cl. C12Q 1/68; C07H 21/04; C07K 14/00

(52) U.S. Cl. 435/6; 530/350; 536/24.3

(57) ABSTRACT

In one embodiment of the present invention, a set of partially degenerate oligonucleotides can be used as standards for monitoring array-feature consistency during manufacturing and for calibrating array experiments. In another embodiment, methods for generating deterministic, fully-characterized sets of partially degenerate sequence standards are provided, in which parameters such as the oligonucleotide-sequence length, the generic sequence string, and the complexity of a set of oligonucleotides can be controlled by a user. Various sets of oligonucleotides with partially degenerate sequences may be combined in order to provide more desirable standards for a variety of array-related uses.

### BACKGROUND OF THE INVENTION

Array technologies have gained prominence in biological research and are becoming important diagnostic tools in the healthcare industry. Currently, array techniques are routinely used to determine the concentrations of particular nucleic-acid polymers in complex sample solutions. Array-based analytical techniques are not, however, restricted to analysis of nucleic-acid solutions, but may be employed to analyze complex solutions of any type of molecule that can be optically or radiometrically scanned and that can bind with high specificity to complementary molecules synthesized within, or bound to, discrete features on the surface of an array.

As with many precision-manufactured items, arrays are generally manufactured in large batches, and therefore need to be examined in a quality-control step in order to ensure consistent quality within a particular batch, and among consecutive batches. Monitoring the quality of arrays includes the assessment of the variance of various parameters and characteristics both among selected arrays of a single batch as well as among selected arrays of different batches. During the manufacturing process, array samples from a given batch need to be rigorously tested to determine whether intended characteristics of a particular array design are present at expected values and levels. For example, each feature of an array containing oligonucleotide probes should have approximately the same number of oligonucleotide probes. In general, the quality of the signal intensity measured for a feature of an array depends on the sequence-specific hybridization properties of a target molecule/probe molecule pair. The hybridization properties of target molecules with respect to probe molecules present within each feature of a given array affect the sensitivity and specificity of probes associated with the feature. A sample set selected from a batch of arrays that are manufactured together in a production run can be routinely evaluated after fabrication to ensure that the arrays exhibit reproducible and consistent ranges of sensitivity and specificity. To evaluate the sensitivity and specificity ranges of a given array, several types of standards have been developed, including spike-in standard RNAs and random n-mer oligonucleotides. There are, however, problems associated with each of these types of standards.

In a spike-in approach, known concentrations of spike-in RNA standards that are complementary to control probes on an array are added to an experimentally-derived sample mRNAs. Generally, such RNA standards contain sequences that are not substantially complementary to experimental probe sequences. Since the RNA standard sequences can hybridize to control probes of control features of an array, signal from control feature to which RNA standard sequences hybridize can therefore be used to determine the corresponding concentrations of experimentally-derived mRNAs in a sample solution to which an array has been exposed. Both standard RNAs and experimentally-derived mRNAs can be labeled with the same types of chromophores under conditions that permit equivalent labeling efficiencies.

The signal intensities for RNA standards are scanned from control features, and the signal intensities for experimentally-derived mRNAs are scanned from non-control features containing experimental probe sequences. By plotting known concentrations of standard RNAs with respect to measured signal intensities for the standard RNAs, a standard curve is established from which the concentrations of experimentally-derived mRNA molecules can be determined. However, a standard curve may not accurately predict the concentrations of mRNA species present in an experimental sample from their respective signal-intensity values. Since the molecules composing the RNA standards are complementary to a small number of sequences selected for only control features on a given array, the RNA standards may be subject to different hybridization kinetics than those exhibited by experimentally-derived sample mRNAs that bind to non-control features, and therefore may provide misleading quantification of sample hybridization signals. In addition, the standard curve may not accurately cover the range of concentrations at which experimentally-derived target mRNAs occur in sample solutions.

Different spike-in standards may be needed for different arrays, in order to prevent inadvertent cross hybridization of standard RNAs with experimental probes of non-control features. Moreover, the processes undertaken to produce spike-in standard RNA molecules are time-consuming and labor-intensive. Large batches of RNA standards are particularly costly to prepare, maintain, and label, and their signal intensities are difficult to reproduce in separate experiments. Although large sets of transcription vectors can be transcribed in vitro to produce standards containing unique sequences, the production of a reproducible set of standards for calibrating multiple experimental samples with variable complexities is costly.

In theory, using labeled, randomly-generated oligonucleotides as standards could resolve some of the problems associated with spike-in RNA standards by providing a diverse set of standard molecules that can hybridize with similar efficiencies to many sequence-specific experimental probes of a given array. Often, more advanced arrays contain in excess of 20,000 distinct sequences. The cost of synthesizing, labeling, and reproducibly blending a large number of distinct oligonucleotides is prohibitively high. Although a single synthesizer run can produce mixtures of random oligonucleotides, as the sequence length of an oligonucleotide is increased to promote optimal hybridization thermodynamics, the concentration of any given sequence decreases precipitously. For reasonable target loadings, it becomes improbable that even a single molecule of a given single sequence is actually present in a sample. Consistent, uniform, and reproducible standard hybridization becomes, as a result, difficult to achieve with longer oligonucleotides.

While spike-in RNA standards and randomly-generated oligonucleotide standards have proved to be useful, quality-control test designers and array manufacturers have recognized the need for standards that can be inexpensively and reproducibly produced, and that can provide deterministic and controllable hybridizations to arrays in order to facilitate quality control over a number of manufacturing runs and to facilitate the calibration of multiple array-based experiments.

### SUMMARY OF THE INVENTION

In one embodiment of the present invention, a set of partially degenerate oligonucleotides can be used as standards for monitoring array-feature consistency during manufacturing and for calibrating array experiments. In another embodiment, methods for generating deterministic, fully-characterized sets of partially degenerate sequence standards are provided, in which parameters such as the oligonucleotide-sequence length, the generic sequence string, and the complexity of a set of oligonucleotides can be controlled by a user. Various sets of oligonucleotides with partially degenerate sequences may be combined in order to provide more desirable standards for a variety of array-related uses.

### BRIEF DESCRIPTION OF THE DRAWINGS

FIG. 1 illustrates general sampling techniques for array quality-control, as one embodiment of the present invention.

FIG. 2 illustrates a conventional method for normalizing differential expression ratios determined for array experimental samples.

FIG. 3 illustrates predicted signal intensities for a set of fully degenerate oligonucleotide standards hybridized to an array for QC evaluation.

FIG. 4 illustrates two sets of oligonucleotides with partially degenerate sequences.

FIG. 5 illustrates two exemplary sets of oligonucleotides with partially degenerate sequences.

FIG. 6 is a flow-control diagram representation of a method for using one or more sets of partially degenerate sequences as a standard for monitoring quality-control procedures that represent embodiments of the present invention.

FIG. 7 is a flow diagram of a method for using one or more sets of partially degenerate sequences as a standard for calibrating array experiments in a two-color experiment that represents one embodiment of the present invention.

### DETAILED DESCRIPTION OF THE INVENTION

In various aspects, the present invention relates to methods for producing sets of partially degenerate sequence oligonucleotides for use as standards during quality-control procedures, and for use in calibrating array experiments. In other aspects, the present invention relates to sets of partially degenerate oligonucleotide sequences generated by a partially combinatorial synthetic scheme. In a first subsection, below, additional information about arrays is provided.

### Molecular Arrays

The present invention is related to arrays. In order to facilitate discussion of the present invention, a general background for particular types of arrays is provided below. In the following discussion the terms “array,” “molecular array,” and “array” are used interchangeably. The terms “array” and “molecular array” are well known and well understood in the scientific community. As discussed below, an array is a precisely manufactured tool which may be used in research, diagnostic testing, or various other analytical techniques to analyze complex solutions of any type of molecule that can be optically or radiometrically detected and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions.

An array may include any one-, two- or three-dimensional arrangement of addressable regions, or features, each bearing particular chemical moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

When an array is manufactured, chemical moieties, such as nucleic-acid molecules, are deposited as probes onto addressable regions of the array surface, or features, by numerous processes. Arrays can be fabricated using drop deposition from pulsejets of either polynucleotide precursor units, in the case of in situ fabrication, or from previously obtained polynucleotides. Such methods are described in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop-deposition methods can be used for fabrication, as previously described herein. Also, instead of drop-deposition methods, known photolithographic array fabrication methods may be used. Interfeature areas, which are areas that do not display probe molecules, need not be present particularly when the arrays are made by photolithographic methods as described in those patents.

The probes bound to the surface of a molecular array are typically exposed to a sample including labeled target molecules, or to a sample including unlabeled target molecules followed by exposure to labeled molecules that bind to unlabeled target molecules bound to the array. The array is then read. Reading of the array may be accomplished by illuminating the array and reading the location and intensity of resulting fluorescent emission at multiple regions on each feature of the array. For example, a scanner may be used for this purpose, which is similar to the AGILENT ARRAY SCANNER manufactured by Agilent Technologies, Palo Alto, Calif. Other suitable apparatus and methods are described in U.S. patent application Ser. No. 10/087,447 “Reading Dry Chemical arrays Through The Substrate” by Corson et al., and Ser. No. 09/846,125 “Reading Multi-Featured arrays” by Dorsel et al. However, arrays may be read by any other method or apparatus than the foregoing, with other reading methods including other optical techniques, such as detecting chemiluminescent or electroluminescent labels, or electrical techniques, in which each feature is provided with an electrode to detect hybridization at that feature in a manner disclosed in U.S. Pat. No. 6,251,685, U.S. Pat. No. 6,221,583 and elsewhere.

A molecular array is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT ARRAY SCANNER manufactured by Agilent Technologies, Palo Alto, Calif. Other suitable apparatus and methods are described in U.S. patent applications: Ser. No. 10/087,447 “Reading Dry Chemical Arrays Through The Substrate” by Corson et al., and Ser. No. 09/846,125 “Reading Multi-Featured Arrays” by Dorsel et al. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from a method disclosed herein may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as signals (e.g., electrical, optical, radio signals, etc.) over a suitable communication channel, for example, over a private or public network. Forwarding an item refers to any means of getting the item from one location to the next, whether by physically transporting that item or, in the case of data, physically transporting a medium carrying the data or communicating the data.

As discussed above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

Deoxyribonucleic acid (“DNA”) and ribonucleic acid (“RNA”) are linear polymers, each synthesized from four different types of subunit molecules. A DNA molecule may be composed of the following subunits: (1) deoxy-adenosine (“A”); (2) deoxy-thymidine (“T”); (3) deoxy-cytosine (“C”); and (4) deoxy-guanosine (“G”). Phosphorylated subunits of DNA and RNA molecules called “nucleotides” are linked together through phosphodiester bonds to form DNA and RNA polymers. A DNA polymer can be characterized by writing a sequence of “A,” “T,” “C,” and “G” single letter abbreviations that represent nucleotide subunits that together compose the DNA polymer. For example, a DNA oligonucleotide can be chemically represented as “ATCG.” Two DNA strands linked together by hydrogen bonds form the familiar helix structure of a double-stranded DNA helix. Double-stranded DNA may be denatured, or converted into single-stranded DNA, by changing the ionic strength of the solution containing the double-stranded DNA or by raising the temperature of the solution. During renaturing or hybridization, complementary base pairs of AT and GC, also known as Watson-Crick (“WC”) base pairs, within anti-parallel DNA strands form in a cooperative fashion, leading to reannealing of the DNA duplex. Single-stranded DNA polymers may be renatured, or converted back into DNA duplexes, by reversing the denaturing conditions; for example, by lowering the temperature of the solution containing complementary, single-stranded DNA polymers. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic-acid polymers, the greater the stability of hybridization between the two polymers under renaturing conditions.

As an example of a non-nucleic-acid-based molecular array, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a molecular array by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of molecular-array data can produce detailed chemical and biological analyses, disease diagnoses, and other information that can be stored in a computer-readable medium, transferred to an intercommunicating entity via electronic signals, printed in a human-readable format, or otherwise made available for further use.

### Quality Control of Manufactured Arrays and Calibration of Array Experiments

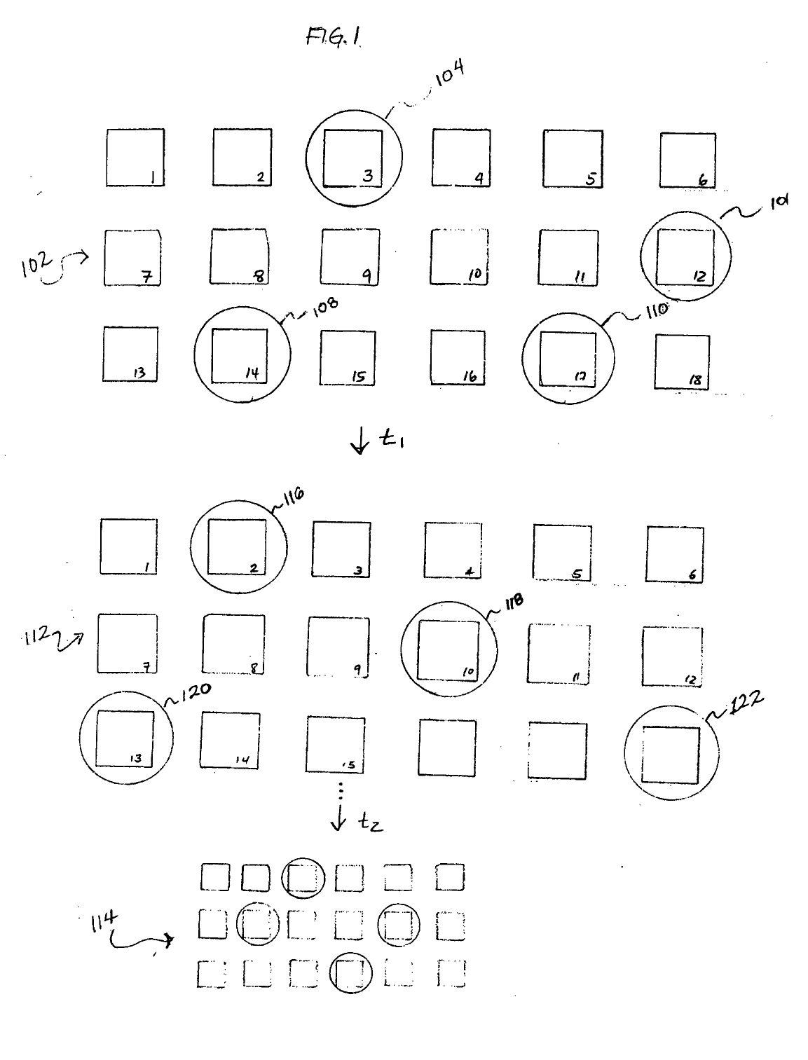

FIG. 1 illustrates general sampling techniques for array quality-control, as one embodiment of the present invention. These techniques involve the evaluation of array samples selected from array batches produced by manufacturing processes in order to assess intra-batch and inter-batch variability during quality-control procedures. In FIG. 1, three batches 102, 112, and 114 of manufactured arrays are shown. Each square box represents a hypothetical array produced in a fabrication run. For each fabrication run, a large number of arrays can be produced. The total number of microarrays in each fabrication run is represented in FIG. 1 with 18 arrays, although, in practice, many hundreds, thousands, or tens of thousands of arrays may be produced in a given fabrication run. After manufacturing the first batch of arrays 102, a first randomly-selected set of test arrays, such as the four arrays 104, 106, 108, and 110, are subjected to destructive quality testing. After manufacturing the second batch of arrays 102, a second randomly selected set of test arrays, such as the four arrays 116, 118, 120, and 122, are subjected to similar destructive quality testing. The consistency of array performance within a single batch of arrays can be assessed by determining a comparison metric, such as a computed statistical variance, with respect to various measured characteristics, of arrays of a single randomly-selected set of test arrays. For example, the consistency of array performance in one batch can be compared to the consistency of array performance in a different batch by comparing the measured variances, with respect to various measured characteristics of arrays of two randomly-selected sets of test arrays. Generally, manufacturers seek to minimize intra-batch and inter-batch variability so that they can provide arrays that produce consistent and reproducible results in multi-array experiments.

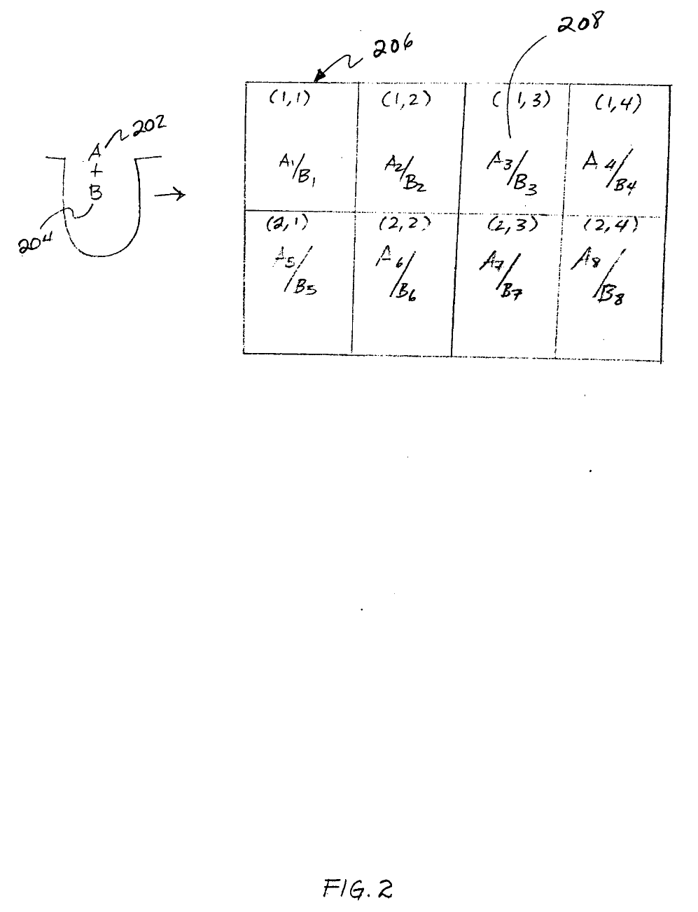

FIG. 2 illustrates a conventional method for normalizing differential expression ratios determined by array experiments. In FIG. 2, two biological samples A 202 and B 204, obtained under different experimental conditions predicted to elicit differential gene expression are hybridized to a hypothetical array, such as array 206. Sample A can be labeled with a first chromophore, and sample B can be labeled with a second chromophore having an emission spectrum distinct from that of the first chromophore. The array 206 can be assayed using a two-channel system to determine signals from an mRNA sample A 202 labeled with a first chromophore, and to determine signals from another mRNA sample B 204 labeled with a second chromophore. For simplicity, only 8 features of a hypothetical array are shown. If the signal intensities derived from a first chromophore can be normalized with respect to the signal intensities derived from a second chromophore, then the differential expression ratios, which are commonly expressed as log ratios of a first-color signal to a second-color signal for each feature, represent the corresponding ratio of the concentrations of mRNA targets that are specific for a feature in an original sample solution. If the log ratio of A and B signals, or log (Aᵢ/Bᵢ), for feature i is 0 or nearly 0, then the gene corresponding to the mRNA target for feature i appears not to be differentially expressed in the two experimental conditions, while log ratios greater than 0 or less than 0 indicate over expression and under expression, respectively, of the genes corresponding to the target mRNAs. When the normalized signals can be scaled or interpreted with respect to the actual amount of a chromophore present within array features, absolute expression levels may be inferred, based on sample dilution and sample preparation metrics. For example, each feature in FIG. 2, can be associated with an expression ratio, such as the expression ratio “A₃/B₃” 208 determined for feature (1, 3) following a feature-extraction step that typically precedes data normalization.

Normalization based on comparative expression ratios determined by such methods may introduce significant errors for various reasons. For example, although experimental conditions may be controlled so that equivalent amounts of two samples are placed in the same solution and exposed to a common array, the measurements of raw signal intensities for sample A and B may not accurately reflect the absolute levels of mRNA contained in each sample. Additional experimental control measures may be warranted. For example, if sample A is first labeled with Cy3, and sample B is labeled with Cy5, then it may be necessary to repeat the experiment reversing the labels used, with sample A labeled with Cy3 and sample B labeled with Cy5 to ensure that differential labeling or differential label-specific signal efficiencies are not responsible for the apparent concentration differences indicated by the experiment. Moreover, when multi-array experiments are employed for monitoring gene-expression levels over a significant time period, it may be difficult to normalize the log ratios obtained at a first time point with respect to log ratios obtained at subsequent time points. For example, although a control solution may be commonly employed as a standard, the control solution may degrade over time, or differing experimental conditions may result in uncontrolled variation in sample solutions, leading to differing levels of cross hybridization and other effects that alter normalization. A set of standard molecules of consistent type and quality can improve normalizations of array data and thereby facilitate the detection of less than optimal arrays and decrease the frequency of normalization errors in array-data interpretation.

Non-Ideal Hybridization Properties of Fully Degenerate Oligonucleotide Standards

In general, methods for producing various types of oligonucleotides containing naturally-occurring and non-naturally-occurring nucleotides by automated chemical synthesizers are well-established. Available methods enable the production of a variable range of chemically-modified oligonucleotides. At one end of a spectrum, a single, pure oligonucleotide product can be produced using a pure sequence synthetic scheme. For example, at each step of a pure sequence synthetic scheme that sequentially incorporates a next desired base at a particular position of an oligonucleotide, a chemical precursor of the desired base is introduced into a synthesizer containing a column of glass microspheres to which the growing oligonucleotides are attached. After stabilizing an initial coupling product and preparing a reactor for the next coupling reaction, the cycle is repeated. Finally, after several cycles of coupling reactions, a pure oligonucleotide product is cleaved from a support. At the opposite end of the spectrum, a large mixture of oligonucleotides can be produced simultaneously via a random synthetic scheme. Random sequences of length n can be prepared by adding an equimolar mixture of all four base precursors at each cycle, for n cycles. Such a reaction scheme results in a mixture of oligonucleotides of all possible sequences of length n.

Both synthetic schemes are inadequate in producing oligonucleotide molecules for use as standards for molecular array-related purposes. Contemporary high-density arrays often contain in excess of 20,000 distinct oligonucleotide sequences as probe molecules, arranged so that each feature contains an approximately equal amount of a distinct probe sequence. For many array-based assays, the probe molecules are produced without a label because experimentally-derived target molecules, such as mRNAs, are labeled prior to hybridization with the probe molecules. For QC purposes, in order to determine the consistency in the number of probe molecules deposited onto various features of an array, labeled standard molecules are hybridized to sample arrays of a manufactured batch. Ideally, when a set of standard molecules are hybridized to features of an array, the set of standards should react with all the features of an array for maximum coverage, and bound standard molecules should produce a signal intensity from each feature proportional to the surface density of probe molecules attached to that feature.

A fully degenerate oligonucleotide (N)ₙ is produced by randomly incorporating any of the possible 4 nucleotides at every position of a particular oligonucleotide. While the possibility of using randomly-generated, fully degenerate oligonucleotide standards initially appears to be promising, since a set of fully degenerate oligonucleotides is expected to bind reasonably consistently and uniformly to different types of probe molecules of an array so that the signal intensities are proportional to the surface density of probe molecules attached to a particular feature. However, the hybridization properties of fully degenerate oligonucleotides can result in undesirable binding behaviors for several reasons. Although a large heterogeneous set of standard molecules, such as randomly-generated sequences, can be produced via a pure-synthetic scheme by synthesizing a large number of distinct oligonucleotides that can be blended together, the costs for the synthesis, labeling, and reproducible blending of such oligonucleotides are economically unfeasible.

Alternatively, in a single run, a synthesizer can produce mixtures of oligonucleotides containing random sequences that do not need to be blended after synthesis. However, in practice, a set of fully degenerate oligonucleotides is inherently diverse with respect to AT/GC contents, which ultimately affects the hybridization properties of each fully degenerate oligonucleotide. In particular, variable AT/GC content will affect the melting temperatures of oligonucleotide standard molecules. The higher the GC content, the higher the melting temperature, or the temperature required to dissociate a hybridized complementary pair. At a given temperature, those fully degenerate oligonucleotide standard molecules containing higher GC content will hybridize to complementary probe molecules with higher affinity than those randomly-generated oligonucleotide standard molecules containing lower GC, or higher AT, content. Since a set of randomly-generated oligonucleotides consists of molecules having variable melting temperatures, adjusting the hybridization temperature does not solve this problem. For example, decreasing the hybridization temperature will increase the probability of intra-molecular hybridizations or self-annealing, especially as the length of the randomly-generated oligonucleotide increases. Although increasing the length of a randomly-generated oligonucleotide is highly desirable to enable sequence-specific hybridizations, or to optimize hybridization thermodynamics, oligonucleotides may need to have maximum lengths of 19-20 nucleotide monomers. Beyond this upper threshold, the concentration of any given randomly-generated sequence decreases quickly, and, for reasonable target loadings, it becomes improbable that even a single molecule of any given single, fully degenerate sequence is actually present in a target sample.

The sequence diversity of a set of fully degenerate oligonucleotides enables broad coverage of most probes on an array. Fully degenerate oligonucleotides can therefore be useful in quantifying the number of probe molecules present within each feature during quality-control procedures. However, in general, a broad range of signal intensities is normally expected in a gene-expression experiment, in which differentially-expressed genes are expected to exhibit significantly different signal levels at two different scanning wavelengths. In such experiments, a first chromophore is used to label mRNAs produced under a first set of experimental conditions, and a second chromophore is used to label mRNAs produced under a second set of experimental conditions. Following two-color scanning and normalization, differentially-expressed genes correspond to target mRNAs that show signal-intensity ratios greater or less than 1. A high-quality array should therefore be capable of producing a large dynamic range of signal-intensities and signal-intensity ratios. Quite often, array defects and array degradation are first noticeable in a decrease in the dynamic range of signal intensities and signal-intensity ratios extracted from the scanned images of arrays. In addition to hybridizing to a majority of features, the desired standards should exhibit a deterministic range of hybridization over array features, rather than a uniform affinity for the features, so that the dynamic range of signal intensities expected in array experiments can be tested and controlled.

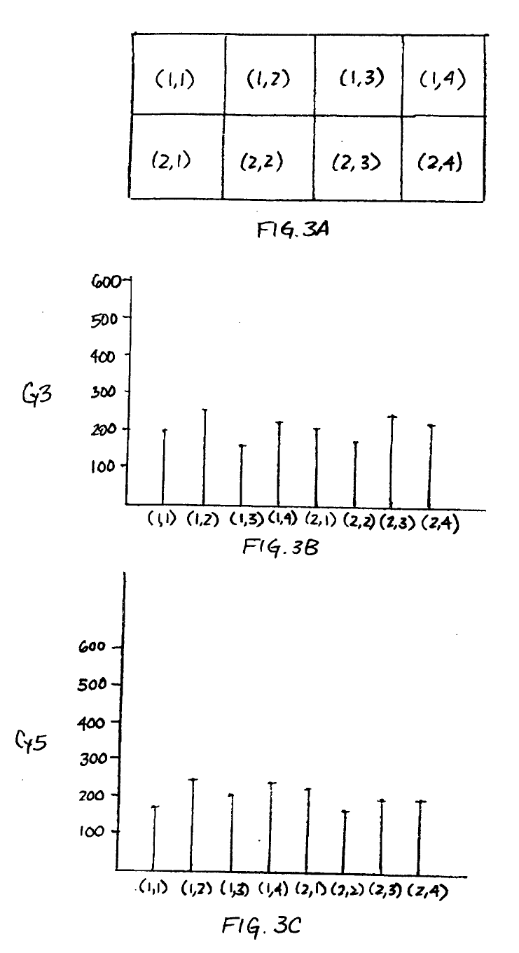

FIG. 3 illustrates hypothetical signal intensities for a set of fully degenerate oligonucleotide standards hybridized to an array for QC evaluation. FIG. 3A illustrates a hypothetical array exposed to two different sets of standards, a first standard set labeled with Cy3, and a second standard set labeled with Cy5. In FIG. 3A, a simple 2×4 array consisting of features (1,1), (1,2), (1,3), and (1,4) in row 1 and features (2,1), (2,2), (2,3), and (2,4) in row 2 is shown as an example. The first fully degenerate oligonucleotide standard set is compositionally distinct from the second fully degenerate oligonucleotide standard set.

FIG. 3B illustrates hypothetical signal intensity values that correspond to binding efficiencies of a Cy3-labeled standard set to array features plotted along the y-axis as a function of feature position. In FIG. 3B, for example, a signal intensity value of 175 is extracted from feature (1,3) when hybridized to a Cy3-labeled fully degenerate standard set. Similarly, FIG. 3C illustrates signal intensity values that correspond to binding efficiencies of a Cy5-labeled fully degenerate standard set to array features plotted along the y-axis as a function of feature position. In FIG. 3C, for example, a signal intensity value of 200 is extracted from feature (1,3) when hybridized to a Cy5-labeled standard set.

In comparing FIGS. 3B and 3C, note that for all the features of the array, including feature (1,3), the signal intensity values corresponding to hybridization activities for Cy3-labeled and Cy5-labeled standards are substantially similar. For example, for feature (1,3), a signal-intensity value of 175 observed when hybridized with the first Cy3-labeled standard set, is different from a signal-intensity value of 200 observed when hybridized with the second Cy5-labeled standard set. Note also that for each signal-intensity profile, a relatively narrow range of intensity values is observed. For example, in FIGS. 3B and 3C, a narrow range of intensity values between 175 and 250 is observed. As explained above, under stringent experimental conditions, only a portion of a set of fully degenerate oligonucleotide standards containing high-GC content is likely to hybridize to a selective set of probes of an array. Broad coverage of the array by high-GC-content, fully degenerate oligonucleotides is not generally obtained. Under less stringent conditions, a set of fully degenerate oligonucleotide standards, such as the example provided in FIG. 3, is likely to exhibit relatively constant hybridization efficiencies for a majority of array features, producing a set of relatively uniform signal intensities rather than a broad range of different signal intensities that is typically observed for experimentally-derived mRNA targets. In other words, a set of fully degenerate oligonucleotide standards exhibits a broad and relatively non-specific affinity for most array features, and does not exhibit a broad range of binding specificities needed to simulate experimentally-derived target binding.

### Partially-Combinatorial Synthetic Methods for the Production of Partially Degenerate Sequence Oligonucleotides

In one embodiment, the present invention provides partially combinatorial synthetic methods for producing oligonucleotides for use as standards in monitoring the quality of arrays during and between manufacturing runs, and for use in data normalization during data interpretation. In one embodiment of the present invention, various sets of partially degenerate oligonucleotide standards can be employed in quality-control procedures designed to assess intra-batch variability, inter-batch variability, array sensitivity, array specificity, and effectiveness of normalization procedures. Maintaining quality control of manufactured arrays is an important step in determining accurate differential expression ratios, since processes that degrade array performance correlate with the degradation of observed expression ratios. In another embodiment of the present invention, various sets of partially degenerate oligonucleotide standards can be employed as calibration tools for facilitating signal extraction, normalization, and scaling. For example, arrays are often used for determining differential expression profiles of genes, a process involving analyzing signals scanned at different wavelengths to detect the relative binding affinities of gene products, each labeled by one of at least two different chromophores.

Synthesis of Partially Degenerate-Sequence Standards

One method for producing a set of partially degenerate oligonucleotide standard molecules includes, at each step of a partially combinatorial oligonucleotide synthesis, a nucleotide precursor solution that may contain equimolar concentrations of a single nucleotide, one of ten possible combinations of two different nucleotides, one of four possible combinations of 3 different nucleotides, or a single possible combination of all four nucleotides, can be employed to add a nucleotide to a growing oligonucleotide.

Table 1 provides a list of standard symbols representing degenerate nucleotide bases used to describe a partially degenerate oligonucleotide sequence. An oligonucleotide may be composed of deoxyribonucleic acid (“DNA”) or ribonucleic acid (“RNA”). For example, a DNA oligonucleotide may be synthesized from four different types of subunit molecules, including deoxy-adenosine (“A”), deoxy-thymidine (“T”), deoxy-cytosine (“C”), and deoxy-guanosine (“G”). A numerical degeneracy is defined as the number of possible nucleotide choices for a particular position in a nucleotide sequence represented as a symbol string. As indicated in Table 1, below, symbols “A,” “T,” “G,” and “C” have degeneracy values of 1. Symbols “R,” “Y,” “M,” “K,” “S,” and “W” have degeneracy values of 2. Symbols “B.” “D,” “H,” and “V” have degeneracy values of 3. Lastly, the symbol “N” has a degeneracy value of 4. Additional non-standard nucleotides and nucleotide combinations may be specified with additional symbols that are not shown in Table 4. Ribonucleic acids include uracil, represented by the symbol “U,” rather than deoxy-thymidine, represented by the symbol “T,” found in deoxyribonucleic acids. Note that a sequence of length n exclusively comprising the symbol “N” specifies a fully degenerate oligonucleotide sequence of length n, so that individual molecules are fully random in sequence, but with a constant, fixed length. A sequence specified with at least one symbol not equal to the symbol “N” is a partially degenerate oligonucleotide sequence, because each symbol not equal to “N” specifies a unique nucleotide or a subset of the 4 possible nucleotides.

TABLE 1

| Symbol | Degeneracy | Nucleotides |
|---|---|---|
| A | 1 | A |
| T | 1 | T |
| G | 1 | G |
| C | 1 | C |
| R | 2 | A or G |
| Y | 2 | T or C |
| M | 2 | A or C |
| K | 2 | G or T |
| S | 2 | G or C |
| W | 2 | A or T |
| B | 3 | C or G or T |
| D | 3 | A or G or T |
| H | 3 | A or C or T |
| V | 3 | A or C or G |
| N | 4 | A or T or G or C |

A set of oligonucleotides with partially degenerate sequences is composed of a family of related sequences that may have identical nucleotide compositions at some positions, and that may vary at certain other positions by incorporating one of a number of alternative nucleotides. Partially degenerate oligonucleotides can be synthesized by employing an automated oligonucleotide synthesizer that adds nucleotide-base-precursor monomers to a growing oligonucleotide, commonly by phosphoramidite-based linking, at each synthetic step. A user can type a sequence string as input to the synthesizer, using specific characters designating the common nucleotides “A,” “,G” “C,” and “T,” as well as additional characters specifying degenerate symbols, such as the character “V” that represents “C,” “G,” or “A,” rather than a single nucleotide, or the character “Y” that represents “C or “T.” A synthesizer can produce equimolar amounts of all possible oligonucleotides having one of the multiple, specified sequences.

In the following, various hypothetical sequences are written in the 3′-to-5′ direction in accordance with combinatorial methods for producing oligonucleotides that initiates the polymerization from the 3′ end of a growing oligonucleotide. Note that this representation is written in an order opposite from the conventional representation in the 5′-to-3′ direction. Thus, for example, a practitioner may enter the sequence “AYGGVT” or “A(T/C)GG(A/C/G)T” into a synthesizer to produce an equimolar mixture of the following oligonucleotides:

ACGGCT
ACGGGT
ACGGAT
ATGGCT
ATGGGT
ATGGAT

Again, these sequences are written in 3′-to-5′ order from left to right.

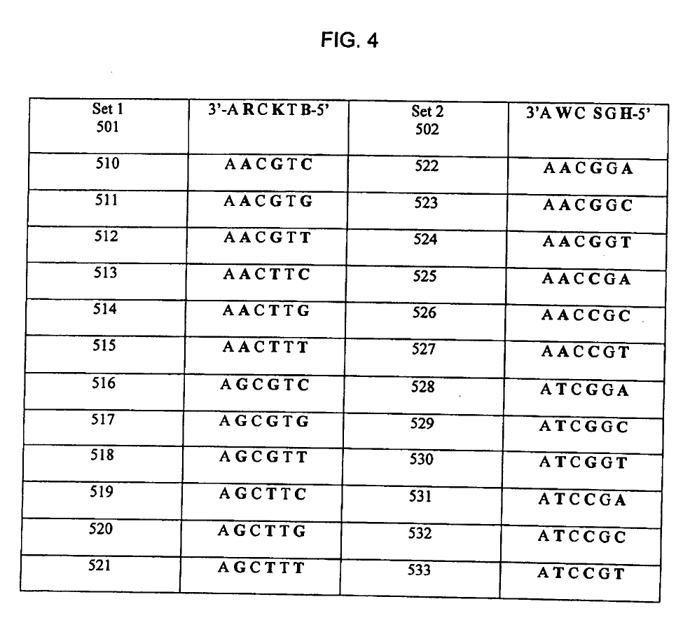

FIG. 4 illustrates two exemplary sets of oligonucleotides with partially degenerate sequences. For simplicity, two sets, “Set 1” 501 and Set 2” 502, of partially degenerate sequences are shown, each set containing 12 distinct sequences. The respective nucleotide positions within a hypothetical sequence are successively numbered from left to right, starting with “A” in position 1 and ending with “T” in position 6. Set 1 501 contains partially degenerate sequences 510-521 defined by the sequence “ARCKTB,” alternatively expressed as “A(A/G) C(G/T) T(C/G/T).” Set 2 502 contains partially degenerate sequences 522-533 defined by the sequence “AWCSGH,” alternatively expressed as “A(A/T) C(G/C) G(A/C/T).” The oligomer sequences of Set 1 and Set 2 are partially degenerate at positions 2, 4, and 6, as shown. As the sequence length of partially degenerate sequences increases, the number of possible distinct sequences increases exponentially, as described further below. In computationally designing sets of partially degenerate oligonucleotide sequences for use as standards, the degenerate nucleotide symbols may be randomly distributed along the entire length of a sequence string, or only within a subsequence of a sequence string. Sets can be mixed together to produce a customized set of standards with desirable hybridization properties with respect to a given set of probes of an array.

The overall complexity of an oligonucleotide sequence is denoted as S. The complexity can be expressed by the equation below, where the variable dᵢ represents the degeneracy of the i^{th} base, and where the number n is the length of the sequence, in nucleotides:

$$S = {\prod }_{i = 1}^{n} {d}_{i}$$

Complexity S is the number of distinct molecules that can be generated from a particular sequence. For convenience, since this number can be quite large for sequences of even very modest length, the logarithm of complexity, or log(S), is commonly used for specifying the degree of complexity for a particular set of partially degenerate oligonucleotides produced by any partially combinatorial synthesis method:

$$log ( S ) = {\sum }_{i = 1}^{n} log ( {d}_{i} ) = log ( {\prod }_{i = 1}^{n} di )$$

A typical array experiment that utilizes an economically feasible quantity of oligonucleotides as target molecules employs between 10⁻⁹ and 10⁻¹³ moles of material, or roughly 10¹¹ to 10¹⁵ molecules. Thus, if the log(S) value for a set of oligonucleotides exceeds about 15, the set of oligonucleotides will generally contain less than one copy of any particular oligonucleotide molecule, so that the presence of a particular oligonucleotide in a sample solution becomes uncertain. For example, a suitable target load should generally consist of at least 10 pmoles of each oligonucleotide per array. For a set of fully degenerate oligonucleotides, a log(S) threshold that guarantees a 10 pmoles concentration for individual oligonucleotides is exceeded when the length of the oligonucleotide in the set is greater than 18-20. In order to be reasonably assured that approximately 10 pmoles of each unique oligonucleotide is included in a standard set of fully degenerate oligonucleotides, the lengths of the fully degenerate oligonucleotides should not exceed 18-20 nucleotides.

In contrast, for a set of partially degenerate oligonucleotides, the log(S) threshold is exceeded at n=25-30. In order to be reasonably assured that approximately 10 pmoles of each unique oligonucleotide is included in a standard set of partially degenerate oligonucleotides, the lengths of the oligonucleotides should not exceed 25-30 bases. Because the maximum length of partially degenerate oligonucleotides within a standard set that provides adequate concentrations of individual oligonucleotide can be greater than the maximum length of fully degenerate oligonucleotide standards in order to achieve adequate sample representation of each type of oligonucleotide, a set of partially degenerate oligonucleotides can be synthesized to have greater average lengths that results in greater average binding affinities for the standard set, while, at the same time ensuring adequate concentrations of individual oligonucleotides.

The concentration of a given partially degenerate oligonucleotide target is the ratio of the overall target concentration to the complexity of the set of target oligonucleotides. For example, if a target set has a complexity, or log (S) value, of 8.5, and the total concentration is 1 μM, then the concentration of any given target species is: (1×10⁻⁶)/10^{8.5}=32 fM (3.2×10⁻¹⁵ M). The hybridization signal expected from such a target set is expected to be greater than that expected based only on the concentrations of individual species because target oligonucleotides having single-base mismatches with respect to particular probes may bind to the probes, and may therefore contribute significantly to the hybridization signal. This effect may increase the concentration of target oligonucleotides by a factor of 10-100. In addition, the hybridization efficiency of a given target oligonucleotide is determined by the driving force for duplex formation and the efficiency of duplex initiation during any given encounter between a probe and a complementary target. Generally, the thermodynamic driving force that drives duplex formation and duplex stability increases with increasing duplex length, or, in the currently considered case, with increasing target length, since the length of a probe is presumed to be greater than the length of a target. The rate of duplex formation can decrease with increasing target length, due mainly to decreased diffusion rates and greater frequency of target self-annealing or self-hybridization. In addition, any tendency of different target oligonucleotides to form duplexes lowers the hybridization efficiency of those species. Therefore, a balance exists between the effect of target oligonucleotide length on hybridization efficiency and the effect of target oligonucleotides length on the concentrations of individual target oligonucleotides in a sample solution. Although theory can guide the proper selection of particular target sets for experimental evaluation, the ultimate choice in selecting one or more target sets of partially degenerate oligonucleotide standards is generally based upon experimental parameters. For example, a reasonable starting point in designing sets of partially degenerate oligonucleotide standards is to produce a standard having a log (S) value of 8.5 and an oligonucleotide length n=17 that exhibits a desirably efficient rate of duplex formation based on complementarity between oligonucleotide target sequences and probe sequences. For a number of additional nucleotides added to a set of oligonucleotides having lengths of 17, the log (S) value of a set of oligonucleotides greater than lengths 17 is calculated by adding log 4 per each additional nucleotide, which is approximately: 8.5+n (0.602). Similarly, for each nucleotide subtracted from a 17-mer, the resulting log (S) value is approximately given by subtracting 0.602 from the log(S) value 8.5 for each nucleotide subtracted.

In partially-combinatorial synthetic schemes, not all possible mixtures can be made in a single synthetic run. For example, in FIG. 4, oligonucleotides that compose Set 1 and Set 2 cannot be produced in a single synthetic scheme if it is desirable that the sequences “AACGTG” 511 and “AACGGA” 522 be included and that the sequence “AACGGG” be excluded. In order to produce the desirable sequences, such as “AACGTG” 511 and “AACGGA” 522, in a single synthetic scheme, both T and G precursors would need to be introduced at step 5 at the fifth position, and both G and A would need to be introduced at step 6 at the sixth position of the growing sequence in the 3′-to-5′ direction. However, introduction of base G at the sixth step produces the undesirable sequence “AACGGG.” Thus, it is not possible to make any arbitrary mixture of oligonucleotides of equal lengths via a single combinatorial synthesis. Two or more independent synthetic schemes may be necessary to achieve desirable results. In this case, Set 1 and Set 2 should be synthesized independently if the undesirable sequence is to be avoided.

Furthermore, certain sequences are inadvertently produced via partially-combinatorial synthetic schemes in the interest of producing particular sequences. For example, if a set of partially degenerate oligonucleotides that include the sequence “XATXX” and “XGCXX” is desired, then a hypothetical sequence string “X(A/G)(T/C)XX” is entered into a synthesizer, where “X” represents any purine or pyrimidine base. A mixture of A and G precursors can be used at the second position and a mixture of T and C precursors can be used for the third position. However, a combinatoric synthetic scheme will unavoidably produce two additional sequences “XACXX” and “XGTXX.” Although certain sequence by-products may be inadvertently produced, as long as such by-product sequences do not adversely affect the hybridization efficiencies of oligonucleotides having the desirable sequences, such as the “XATXX” and the “XGCXX” described above, a partially combinatorial synthesis is a useful approach for producing custom oligonucleotide mixtures that can be used as standards in micoarray-based experiments and QC applications. A set of partially degenerate oligonucleotide standards that generates a range of signals that mimics the range of signals generated by real experimental samples, such as mRNA targets, and that allows statistical calibration and quality control across an entire range of measurement, can be produced by embodiments of the present methods.

Sets of combinatorial sequences having substantial complexity (high S value) can be economically and reproducibly manufactured by using any conventional automated oligonucleotide synthesizer capable of performing combinatorial sequence syntheses. Thus, a combinatorial set of partially degenerate target sequences can be reproducibly manufactured so that the complexity of a particular set of partially degenerate oligonucleotide standards can be consistently controlled. In general, provided that the probe-sequence length exceeds the target-sequence length, as the lengths of probe sequences and degenerate target sequences increase, the ability of a partially degenerate target sequence to generate a spectrum of binding efficiencies to probes on a given array also increases. The spectrum can exhibit a dynamic range in hybridization intensities for both a single-color channel and the differential expression ratios computed for two-color channels.

Partially degenerate oligonucleotide standard sets of the present invention may not only be produced by a single synthesizer-facilitated combinatorial synthesis, but may also be produced as mixtures of independently produced distinct sets of partially degenerate oligonucleotides. For example, to produce a customized set of standard oligonucleotides with sequences and hybridization properties of interest, a small number of distinct sets of partially degenerate sequence standards can be produced by an combinatorial synthesis method and can be blended or mixed together to produce custom mixtures of oligonucleotides that cannot be made via a single-synthesis scheme. Unlike the production of fully degenerate oligonucleotides, a partially combinatorial synthetic method for the production of partially degenerate oligonucleotide sequences is deterministic, resulting in a set of well-characterized standards. The partially degenerate sets and mixtures of such partially degenerate sets can be deterministically and reproducibly manufactured. These sets and mixtures of sets can be evaluated by automated computational techniques to predict binding patterns that result from hybridizations between oligonucleotide standards and probe molecules, cross-hybridizations that result from annealing between complementary sequences of distinct partially degenerate oligonucleotides of a set, self-hybridizations that result from intramolecular, self-annealing between complementary sequences within individual oligonucleotides of a set, and other characteristics. Well-known symbolic, thermodynamic-based, combinatorial hybridization simulation methods may be employed in order to model the hybridization of particular standards to particular arrays to model cross-hybridization of particular standards with a particular, expected sample solution, and to model self-hybridization of particular standards.

Hybridization Properties of Partially Degenerate-Sequence Standards

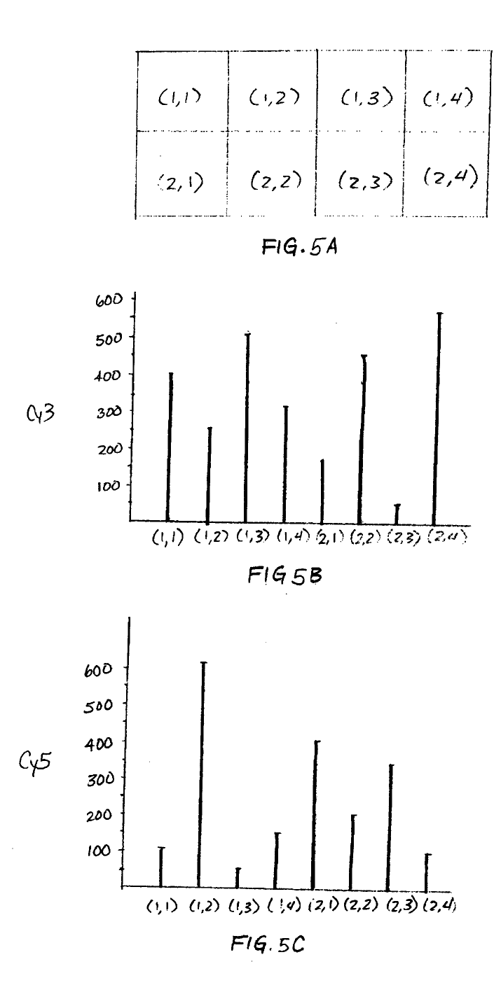

FIGS. 5A-C illustrate properties of partially degenerate oligonucleotide standards that can be used in array applications according to one embodiment of the present invention. For monitoring quality-control in manufactured arrays, a suitable set of standards should collectively react with most probes of an array in order to maximize probe coverage. However, as discussed above, an ideal set of standards should exhibit varied binding affinities for different probe molecules of the array so that a full dynamic range of an array, and, in multiple-color experiments, should provide a broad range of signal ratios mirroring the ranges of signal-intensity ratios that might occur as a result of signals produced by differentially-expressed genes. The partially degenerate oligonucleotide standards of the present invention can be labeled with a variety of fluorescent, chemiluminescent, or other reporters that are known in the art.

FIG. 5A illustrates an array exposed to two different sets of standards: (1) a first standard set labeled with Cy3, and (2) a second standard set labeled with Cy5. In FIG. 5A, a 2×4 array consisting of features (1,1), (1,2), (1,3), and (1,4) in row 1 and features (2,1), (2,2), (2,3), and (2,4) in row 2 is shown. The first partially degenerate oligonucleotide standard set is compositionally distinct from the second partially degenerate oligonucleotide standard set. FIG. 5B illustrates signal-intensity values that correspond to binding efficiencies of a Cy3-labeled, partially degenerate standard set to array features. In FIG. 5B, for example, a signal-intensity value of 500 is obtained from feature (1,3) when hybridized to a Cy3-labeled standard set. FIG. 5C illustrates signal intensity values that correspond to binding efficiencies of a Cy5-labeled partially degenerate standard set to the array features in the same manner as FIG. 5B. In FIG. 5C, for example, a signal intensity value of 50 is extracted from feature (1,3) when hybridized to a Cy5-labeled standard set.

In comparing FIGS. 5B and 5C, note that, for all the features of the array, including feature (1,3), the signal intensity values corresponding to hybridization affinities for Cy3-labeled and Cy5-labeled partially degenerate standards are substantially different. For example, for feature (1,3), the signal-intensity value of 500 observed when feature (1,3) is hybridized with the first Cy3-labeled partially degenerate standard set is different from the signal-intensity value of 50 observed when feature (1,3) is hybridized with the second Cy5-labeled partially degenerate standard set. Note also that, for each signal-intensity profile, a broad range of intensity values is observed. For example, in FIGS. 5B and 5C, a broad range of intensity values between 50 and 600 is observed. In the example provided in FIGS. 5B and 5C above, two different partially degenerate standard sets show varied binding affinities for different probe molecules resulting in a broad range of hybridization affinities for different features, and the binding affinities are different for each standard set leading to a corresponding broad range of observed signal-intensity log ratios. The signal-intensity ratios for the Cy3-labeled and Cy5-labeled partially degenerate standards exhibit a broad range of values, but the signal-intensity ratio is deterministically predictable for each feature.

In one embodiment, a set of partially degenerate oligonucleotide standards, such as the Cy3-labeled standards and Cy5-labeled standards described in FIGS. 5B and 5C, can be produced in equimolar amounts of each partially degenerate oligonucleotide sequence. In some situations, equimolar concentrations of partially degenerate oligonucleotide standards can serve as ideal array standards, particularly when the partially degenerate oligonucleotide standards can be designed to produce particular binding affinities with respect to particular features, and to exhibit minimal cross hybridization with other partially degenerate oligonucleotide standards and predicted experimentally-derived targets. In addition, by using a defined set of reproducibly manufactured, partially degenerate oligonucleotide standards, a manufactured array from any batch, made at anytime, can be assayed to evaluate its performance against a particular quality standard established for a particular array.

In another embodiment of the present invention, a set of partially degenerate oligonucleotide standards, such as the Cy3-labeled standards and Cy5-labeled standards described in FIGS. 5B and 5C, can be produced with unequal amounts of each of the partially degenerate oligonucleotides. Thus, the relative quantity of nucleotide precursors available during a coupling reaction can be programmed into an oligonucleotide synthesizer by a practitioner in order to introduce an additional level of variability in producing a customized set of standard oligonucleotides. The synthesis can be further skewed by using mixtures in which the mole fractions of nucleotide precursors are available in unequal amounts. Nucleotide precursors present at higher concentrations will have a proportionately higher probability for incorporation into a growing sequence chain.

In one aspect of the present invention, the partially degenerate oligonucleotide standards, such as the exemplary standard sets discussed above, are deterministic. A particular set of partially degenerate oligonucleotide standards are produced by partially combinatorial methods, described below, to contain known amounts of particular oligonucleotides that can be reproducibly prepared. The oligonucleotides can be used to monitor quality-control over a continuously produced set of manufacturing runs, or to calibrate multiple array-based experimental results, such as multiple results obtained over various time periods. In another aspect of the present invention, the partially degenerate oligonucleotide standards are well-characterized with regard to oligonucleotide composition and concentration. In another aspect of the present invention, a set of such oligonucleotide standards exhibits binding affinity for a majority of different probe molecules of an array, and varied binding affinities for different probe molecules of an array, for which partially degenerate oligonucleotide standards of the set exhibit: (1) minimal cross-hybridization with other partially degenerate oligonucleotide standard molecules of the set so that effective concentrations of cross-hybridizing standard molecules are not reduced; (2) minimal self-hybridization that results from intramolecular, self-annealing between complementary sequences within individual oligonucleotide molecules of the set; and (3) minimal cross-hybridization with experimentally-derived sample target molecules. Different standards may be needed for quality-control and calibration of different arrays, and therefore, in one aspect of the present invention, standards are computationally evaluated for coverage and differential-binding affinities to features of an array.

Methods for Quality Assessment of Manufactured Arrays

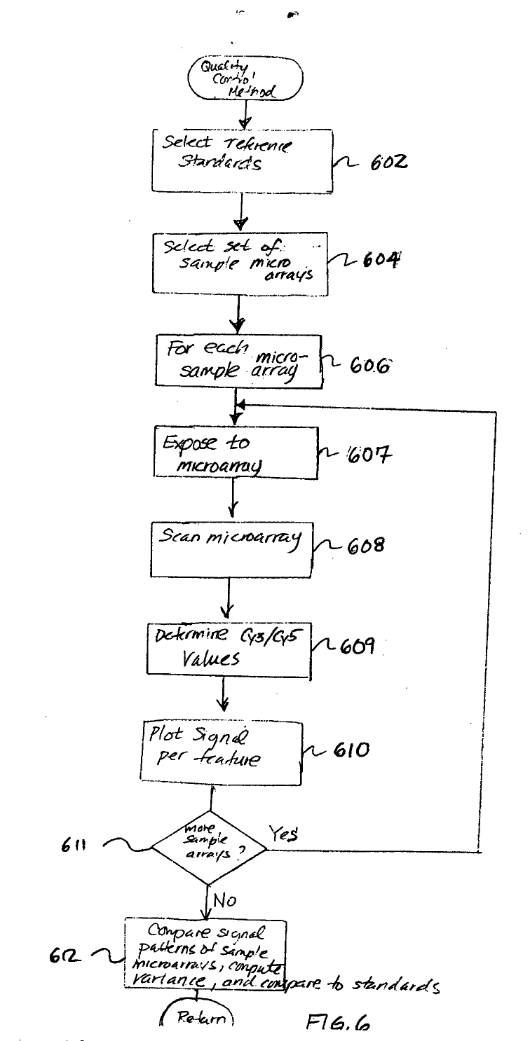

For the characterization of arrays, a partially degenerate oligonucleotide standard comprising a single partially degenerate oligonucleotide set, or a mixture of partially degenerate oligonucleotide sets, can be hybridized to a test array, and the resulting pattern of signal intensities can be analyzed to detect systemic deficiencies. FIG. 6 is a flow-control diagram representation of a method for using one or more sets of partially degenerate sequences as a standard for monitoring quality-control procedures that represents one embodiment of the present invention. In step 602, two partially degenerate sequence standards are selected, based on a computational analysis, prior experience, and other considerations. Each of the two standards are labeled with a different chromophore or radiolabel and then combined to form a single standard. In step 604, a set of sample arrays is selected from a batch of manufactured arrays. In the for-loop comprising steps 607-611, the sample arrays are exposed to a set of standards containing a particular concentration of partially degenerate oligonucleotide sequences. The resulting signal intensities that are observed for each sample array are plotted, displayed, or quantified, and stored as data sets. In step 612, the observed signal intensities are employed to determine a comparison metric, such as statistical variances, for the sample arrays and to compare the observed signal intensities with expected signal intensities, or standard signal intensities, in order to infer the consistency and the quality of the batch of arrays from which the sample arrays are selected.

As one embodiment of the present invention, one or more sets of partially degenerate sequences may be used in conjunction with other tests that are sensitive to known error modes, such as periodic decreases in array-column intensity often associated with defects in the array-printing apparatus. As another embodiment of the present invention, one or more sets of partially degenerate sequences may be used in conjunction with tests that compare the observed hybridization pattern to a historical record or to an average. These tests are empirical, and such tests monitor both the hybridization intensities and channel intensity ratios for multi-color arrays. Global and local degradation of either or both measurements indicates a degradation in array quality, such as a decrease in probe integrity. For example, low concentrations of probe oligonucleotide in the input printing solutions can result in reduced amounts of array probe molecules, which can reduce the dynamic signal range and provide an underestimation of differential expression.

Improvements in quality control enabled by partially degenerate oligonucleotide standards of the present invention can result in improved array quality, and can reduce array fabrication costs. Reduction in the costs for evaluating the quality of arrays produced may be attributed to an increase in hybridization rates of partially degenerate oligonucleotides in comparison to lower hybridization rates that are necessary for the binding of longer target molecules, such as cDNAs derived by in vitro transcription and fully degenerate oligonucleotides, at comparable concentrations.

Methods for Calibrating Array Experiments

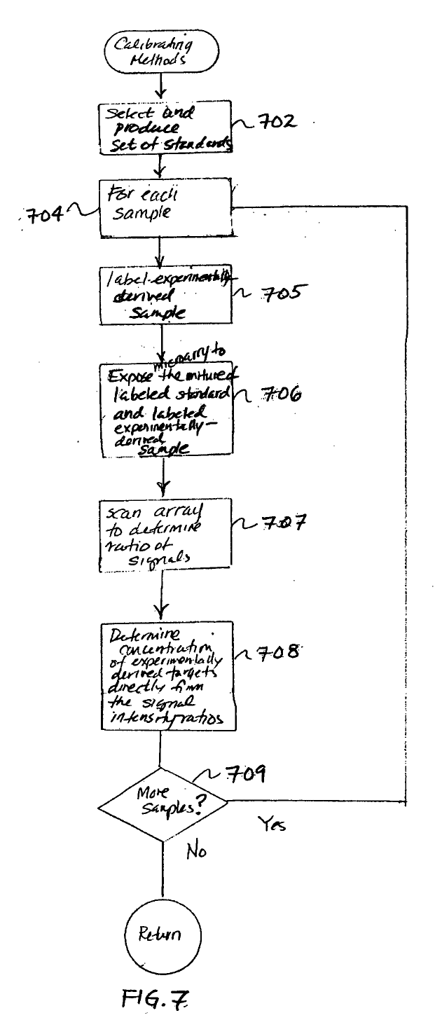

One or more sets of standards composed of partially degenerate oligonucleotides, obtained by the disclosed methods or by various alternative methods, can be used for scaling the signal-intensity-based target concentrations inferred from multiple-array analysis of multiple samples. FIG. 7 is a flow diagram representation of a method for using one or more sets of partially degenerate sequences as a standard for calibrating array experiments in a two-color experiment that represents one embodiment of the present invention. The differential gene expression ratios obtained by detecting different levels of gene products, such as mRNAs, in multiple tissue solutions, can be determined as follows. In step 702, one or more sets of partially degenerate oligonucleotide sequences are selected and corresponding oligonucleotides produced based on a computational analysis, prior experience, for other reasons, or a combination thereof. In the for-loop of steps 704-709, a two-color array analysis is performed on each sample solution. In step 704, an experimentally-derived sample solution to be evaluated is obtained, and is labeled by various methods in step 705. In step 706, the labeled, partially degenerate oligonucleotide standards are mixed with the labeled, experimentally-derived sample solution and an array is exposed to the resulting mixture. In step 707, the array is scanned to obtain a set of signal-intensity ratios for each feature of the array. In step 708, the concentrations of experimentally-derived targets in the sample solution can be directly calculated from the observed signal-intensity ratios for features corresponding to the experimentally-derived targets, since the observed signal-intensity ratios, once normalized, essentially represent normalized signal intensities. Because the same set of partially degenerate oligonucleotide standards is employed to analyze each sample solution, the determined target concentrations for each sample solution are all automatically normalized to a common reference sample.

A set of partially degenerate oligonucleotide standards of the present invention can be applied in various contexts, involving the evaluation of any number of comparative samples, including samples collected over a multiple-point time course, samples treated with variable concentration of agents, and samples of biopsies from multiple donors. Furthermore, because these methods allow a user to consistently label a standard set using the same label, such as Cy3, these methods also permit the consist labeling of all samples to be evaluated with a different label, such as Cy5, in order to prevent the introduction of chromophore-related variance in the normalization of signal-intensity data.

In a related embodiment, a biological reference sample “R” can be pre-established for a given array type, and the performance of the reference sample can be established relative to a standard set of partially degenerate oligonucleotides “C.” If the average performance is denoted as {R/C}, then the performance of a sample of interest “A” relative to R can be calculated by the equation: A/R=(A/C)/{R/C}.

Incorporation of Chemically-Modified Bases into Partially Degenerate Oligonucleotide Standards

The partially degenerate oligonucleotide standards of the present invention can be modified by incorporating chemically-modified bases during synthesis using any of the conventional methods known to persons skilled in the art. Such modifications that increase the melting temperature of partially degenerate oligonucleotides may be desired in order to improve target binding thermodynamics. In one embodiment of the present invention, a set of partially degenerate oligonucleotide standards can be produced by incorporating various modified bases that result in various types of unstructured nucleic acids (“UNA”) containing non-natural nucleotides, including locked nucleic acids (“LNA”), or alternatively referred to as bridged nucleic acids (“BNA”). In general, UNA oligonucleotides do not exhibit self-annealing structures and duplex formation between different sequences of a set of oligonucleotide standards. Such self-annealing structures and intermolecular duplex formation among components of a standard set would suppress duplex formation between the UNA oligonucleotide target standards and complementary probe oligonucleotide. LNA oligonucleotides contain LNA oligonucleotides composed of ribonucleotide analogs with a methylene linkage between the 2′ oxygen and the 4′ carbon of the ribose ring. A conformational constraint on the sugar moiety results in a locked 3′-endo conformation that preorganizes base for hybridization resulting in an increase in melting temperature as much as 10° C. per base. Because of the enhanced thermodynamic stability of LNA oligonucleotides, LNA oligonucleotides with hybridization properties comparable to naturally-occurring oligonucleotides of one length range may be produced at shorter lengths. Thus, incorporation of LNA oligonucleotides into an oligonucleotide sequence may be desired, especially when the sequence length needs to be decreased. LNA oligonucleotide standards may form stable hybrids with both RNA and DNA oligonucleotide probes. Methods for designing and producing various UNA and LNA oligonucleotides are known to persons skilled in the art. In general, as much as 95% of an oligonucleotide sequence may be composed of UNA oligonucleotides, and most of the sequence may be composed of LNA oligonucleotides if desired. By either approach, UNA and LNA oligonucleotides exhibit substantially increased propensity for duplex formation, which can be desirable when designing sets of standards for evaluating arrays and normalizing array-based results. Tolstrup et al., “Oligo design: optimal design of LNA oligonucleotide capture probes for gene expression profiling,” Nucleic Acids Research, Vol. 31, No. 13, 3758-3762 (2003); Kurreck et al., “Design of antisense oligonucleotides stabilized by locked nucleic acids,” Nucleic Acids Research, Vol. 30, No. 9, 1911-1918 (2002); and Nielsen et al., “NMR structure of an alpha-L-LNA/RNA hybrid: structural implications for RNAse H recognition,” Vol. 31, No. 20, 5858-5867 (2003). U.S. Patent Application Publication 20040086880 by Jeffrey Sampson, Robert Ach, and Paul Wolber is incorporated by reference.

In another embodiment of the present invention, a set of partially degenerate oligonucleotide standards containing peptide nucleic acids (“PNA”) of various lengths can be produced. In general, PNAs exhibit higher melting temperatures than standard oligonucleotides, and can be useful when partially degenerate oligonucleotide standards having enhanced thermostability or shorter sequence length are desirable. PNAs may be designed and synthesized by various methods known to persons skilled in the art, including solid-phase methods.

Although the present invention has been described in terms of particular embodiments, it is not intended that the invention be limited to these embodiments. Modifications within the spirit of the invention will be apparent to those skilled in the art. For example, although the described embodiments relate to oligonucleotide-based standards and experimentally-derived target, the approach of the present invention may be applied to a variety of target and reference-standard molecules that bind to probe molecules based on sequence-specific characteristics, such as protein targets that bind to DNA probes. The technique can be applied to two channel, or multi-channel experiments using multiple types of labels or moieties, including different radiolabels, fluorophore, chemiluminescent, and antibody labels, and other types of chemically or instrumentally distinguishable molecular labels, such as mass tags. The partially degenerate standards may be produced via synthesizers, as discussed above, or by various other methods, including different chemical, chemical/mechanical, chemical/electrical, and chemical/electrical/mechanical methods. Although the discussed synthesizer-based technique generates equimolar mixtures, non-equimolar mixtures generated by other techniques may be employed, provided that the concentrations of each different type of molecule are known.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A standard for array calibration and for array quality-control comprising a set of oligonucleotide molecules having partially degenerate sequences and having a complexity, log (S), less than 11.

2. The standard of claim 1 wherein the complexity is less than 15.

3. The standard of claim 1 wherein the set of oligonucleotide molecules includes a combination of two or more subsets of partially degenerate sequences, each subset independently produced.

4. The standard of claim 1 wherein the oligonucleotide molecules are composed of at least one of:
deoxyribonucleic acids (DNA);
ribonucleic acids (RNA);
locked nucleic acids (LNA);
bridged nucleic acids (BNA);
unstructured nucleic acids (UNA);
peptide nucleic acids (PNA); and
derivatives of DNA, RNA, LNA, BNA, UNA, and PNA.

5. The standard of claim 1 wherein the oligonucleotide molecules are labeled by one of:
a chemiluminescent moiety;
a moiety including a mass tag; and
a moiety including a radioisotope.

6. A method for preparing a standard set of oligonucleotides useful for calibrating arrays and for monitoring quality-control of arrays comprising:
selecting one or more sets of oligonucleotides, each set of oligonucleotides specified by a symbol string;
for each symbol string, inputting the symbol string into a synthesizer to produce a set of oligonucleotide molecules having partially degenerate sequences specified by the symbol string; and
when two or more sets are produced as specified by two or more symbol strings, combining the two or more sets of oligonucleotides to produce a standard set of oligonucleotides.

7. The method of claim 6 wherein selecting one or more sets of oligonucleotides further comprises employing computational techniques in order to select a set of oligonucleotide standards that
binds to a majority of different probe molecules of the array; and
binds with varied affinities to different probe molecules of the array.

8. The method of claim 6 wherein the set of oligonucleotide standards comprises partially degenerate oligonucleotide standard molecules that
minimally cross-hybridize with other partially degenerate oligonucleotide standard molecules of the set;
minimally self-hybridize; and
minimally cross-hybridize with experimentally-derived sample target molecules.

9. A method for evaluating a batch of manufactured arrays comprising:
exposing a subset of test arrays selected from the batch of manufactured arrays to a standard set of oligonucleotides having partially degenerate sequences;
determining a set of signal intensities for each test array;
comparing the determined signal intensities for the test arrays to determine a comparison metric; and
rejecting the batch of manufactured arrays when the determined comparison metric exceeds a threshold value.

10. The method of claim 9 wherein the comparison metric is derived from a statistical variance computed for the signal intensities of the test arrays.

11. The method of claim 9 wherein the comparison metric is derived from a statistical difference between the signal intensities measured for the test arrays and a standard set of signal intensities.
